(19) United States
(12) Patent Application Publication
(10) Pub. No.: US 2010/0286790 A1
(43) Pub. Date: Nov. 11, 2010

(54) IMPLANT AND METHOD FOR COATING AN IMPLANT

(75) Inventors: Heiko GRUNER, Beinwil am See (CH); Philipp Gruner, Villmergen (CH); Francis Tourenne, Belfort (FR)

Correspondence Address: BURR & BROWN, PO BOX 7068, SYRACUSE, NY, 13261-7068, US

(73) Assignee: Medicoat AG, Magenwil (CH)

(21) Appl. No.: 12/778,419

(22) Filed: May 12, 2010

Related U.S. Application Data
(63) Continuation of application No. PCT/EP2008/009519, filed on Nov. 12, 2008.

(30) Foreign Application Priority Data
Nov. 12, 2007 (DE) 10 2007 054 214.5

(51) Int. Cl. A61F 2/28; B05D 3/10; C04B 28/34; C04B 12/02; B32B 9/00

(52) U.S. Cl. 623/23.6; 427/2.24; 106/690; 106/18.31; 428/701

(57) ABSTRACT

The invention relates to an implant made of biocompatible materials, in particular a prosthesis implanted without cement for traumatology and/or orthopedics, which has a main body with an anchoring region which anchors in bone or tissue, with the anchoring region being provided at least partially with a covering layer, the covering layer being formed from a powder using a thermal spraying method, in particular a plasma spraying method. The powder consists essentially of calcium phosphate and comprises antibacterially effective active constituents.

### CROSS REFERENCE TO RELATED APPLICATIONS

This application is a continuation of International Application No. PCT/EP2008/009519 filed Nov. 12, 2008, which designated the United States, and claims the benefit under 35 USC §119(a)-(d) of German Application No. 10 2007 054 214.5 filed Nov. 12, 2007, the entireties of which are incorporated herein by reference.

### FIELD OF THE INVENTION

The invention relates to an implant made of biocompatible materials, in particular a prosthesis implanted without cement for traumatology and/or orthopedics and a covering layer formed from a powder using a thermal spraying method, in particular a plasma spraying method.

### BACKGROUND OF THE INVENTION

Since 1985, artificial joint parts have been mass-produced with a calcium phosphate layer applied by various methods of thermal spraying, for quick, forced osseointegration in the surrounding bone tissue. This technology dates back to a Japanese Patent Application No. 50-158745 A1 (Sumitomo Chemical Co.) and has been continually optimized over the years for solving the stated problem. At present, vacuum plasma spraying (VPS) is mainly used for forming the layer. This method makes it possible for the calcium phosphate powder particles that are melted for forming the layer to be kept above the required melting temperature for just a short time, so that critical phase transformations are minimized during resolidification, and nevertheless a mechanically stable, firmly-adhering layer of a ceramic nature is formed. The precise process operations and optimization steps are for example described in detail in the section “Thermal Sprayed Coatings on Titanium” in the book “Titanium in Medicine”, ISBN 3-540-66936-1, Springer-Verlag Berlin.

Calcium phosphate layers are used in prosthetics in two different applications, which differ by the desired dwell time of the implant in the human body.

In traumatology, in which an implant assists the healing of bone fractures for a limited time, the calcium phosphate layer should possess a defined solubility and/or mechanical stability for a limited time. Thus, on the one hand it firstly ensures perfect mechanical fixation in the bone/layer assembly, important for rapid union of the fractured bone parts on the contact surfaces that are adjusted to one another. On the other hand this support should be continually lost again through the slow disappearance of the calcium phosphate layer in parallel with the fracture healing process, so that at the end of the envisaged dwell time, these osteosynthesis implants can easily be explanted again.

In orthopedics, whenever an implant that remains in the body permanently is required (so-called endoprostheses), the calcium phosphate layer should be of such a structure that once the direct bone/layer composite has formed, it remains stable throughout the dwell time of the implant in the body and at every movement it provides direct transmission of loading into the implant.

Although owing to this calcium phosphate “magic hat” coating, the artificial implant is no longer recognized as a foreign body by the surrounding bone, rejection occurs in 2-5% of operations. The implant bed in the bone tissue becomes inflamed. The most probable cause is a bacterial infection, which either reaches a critical extent immediately after implantation, or is not manifested until later. The germs that were picked up or were already present in the body are kept at bay in the initial phase of the healing process because of the usual medication, but become active without restraint after the active substances used for assisting wound healing are discontinued, or as a result of an infectious disease. If a subsequent infection develops at the bone/prosthesis interface, administration of antibiotics is usually no longer helpful, as hardly any amount reaches this site of inflammation. In the vicinity of the prosthesis, the tissue is scarred and poorly perfused. Further surgery because of inflammation and rejection is very stressful for the patient and causes considerable costs and economic losses globally, with an upward trend owing to the increasing spread of bacteria that are multiresistant to antibiotics. It is therefore necessary to look for solutions for effective prevention of early or delayed infections and rejection reactions of prostheses that are implanted without cement.

Metals such as silver, but also zinc or copper, have long been known to have antibacterial action. Already in antiquity, silver (Ag) shavings were added to wound ointments, for example. Silver threads incorporated in the gauze dressing for severe burns effectively prevent inflammation due to bacterial action. The diameter and spacing of the Ag threads are selected so that they prevent the migration and penetration of bacteria and viruses. Ag-coated textile fibers for this application have also recently become available. As with all metallic active substances, so too with Ag it is a matter of using the correct dosage: too much damages the human body and too little means the dose has no effect. The concentration of metal ions is decisive for antibacterial efficacy. Silver ions (Ag+), for example, can arise through dissolving-out from metallic silver (Ag0), or can already be present in ionic form. Information on the correct dosage of metal ions is given in the specialist literature, including for the particular application of endoprostheses. However, the figures for dosage given in the literature sometimes differ considerably.

A constant stable antibacterial action is achieved for example with thin layers of silver produced by physical or chemical deposition from the vapor phase in vacuum, but also with layers of silver applied electrochemically. These layers of silver, as described for example in U.S. Pat. No. 7,018,411 B2, “Endoprosthesis with Galvanised Silver Layer,” despite biocompatibility, do not exhibit an osseoconductive or osseoinductive action (E. Sheehan et al., European Cells and Materials, Vol. 10 Suppl. 2, (2001) page 75). Nevertheless, Ag-coated prostheses have been used with excellent antibacterial success in tumor patients at the Munster Hospital since 2005. The short-term prevention of inflammation is more important than the longer-term stable anchorage in this at-risk group of patients.

The antibacterial action of silver nanoparticles has also been investigated and described (H. Y. Song et al., European Cells and Materials, Vol. 11 Suppl. 1, (2006) page 58). The particles, with a size of about 5 nm, display pronounced antibacterial behavior and are recommended as an alternative or supplement to antibiotics. If they are combined with highly porous Ag particles in the size range 2 to 10 μm and incorporated at a concentration of 1% in the bone cement for cemented implantation, these Ag nanoagglomerates provide pronounced antibacterial action even against resistant germs.

Moreover, at the 53rd Annual Meeting of the Orthopaedic Research Society, Ghani J. et al. presented a report on electrochemically deposited layers of hydroxyapatite, which provide controlled release of Ag ions. Unfortunately the antibacterial action achieved only lasts for 6 days, after which release, from the surface of the layer, of the Ag ions incorporated during production of the layer is exhausted. Incorporation of the Ag ions (doping) takes place simultaneously with the electrochemical deposition of the HA layer. The Ag ions are either located between the HA crystallites or are incorporated directly in the HA crystal lattice in place of calcium ions. Consequently, release of the Ag ions is only possible on dissolution of the HA layer, or through diffusion effects.

The use of a sol-gel method and an additional thermal tempering process for producing crystalline HA layers with variable content of Ag, from which release of Ag ions is at first very rapid at high concentration, but then takes place over a longer period, steadily decreasing, is also known (W. C. Chen et al., Key Engineering Material, Vols 330-332 (2007), page 653 to 656).

For antibacterial filter media, porous HA ceramics with incorporated Ag ions have been developed, incorporation taking place by ion exchange during treatment of the porous ceramic material in a 0.2 mol. % AgNO₃ solution for about 1 hour.

U.S. Pat. No. 5,009,898 (1989), which describes an antibacterial calcium phosphate powder and the method of production thereof, is also interesting in this context. Apart from the content of organic antibacterial active substances, e.g. protamine, there is also reference to the antibacterial action of incorporated metal ions, in particular silver ions. At the same time, there is a long list of references, all of which played a role in the granting of the aforementioned patent and thus document the prior art. The metal content in the calcium phosphate and here in particular in the variant of the material hydroxyapatite (HA) is stated with a very wide range from 1 ppm to 50000 ppm. According to the method described, this metal ion-doped HA is precipitated from an aqueous solution, in which a silver salt is dissolved, along with calcium and phosphate. The decisive patent claim is that the silver ions in the HA molecule replace calcium ions in a targeted manner and/or are incorporated as individual ions at interstitial sites of the apatite crystal lattice. As a result, this antibacterial HA modified in this way can additionally take up further antibacterial substances e.g. of a synthetic or organic nature by absorption, without the two active substances affecting one another adversely. Use of this antibacterial HA powder in foodstuffs, cosmetics, in cellulose and, among other things, also in the human body (for example in dentistry) is envisaged, namely whenever antibacterial action is required.

### SUMMARY OF THE INVENTION

The problem to be solved by the invention is to propose an implant or a method for coating an implant or a covering layer for an implant, which combines the advantageous properties of a calcium phosphate layer, in particular a hydroxyapatite layer, for rapid union of the bone tissue with the implant, with a significant decrease in operative and postoperative risk of infection, without adversely affecting the process of union.

For the use of prostheses implanted without cement, their secure anchorage in the patient's bone tissue is of primary importance. The coating used should therefore have unrestricted rapid osseoconductive and osseoinductive action. The intensive bone/prosthesis composite should remain optimal both in the short term and/or in the longer term. Without impairing these properties, bacterial infections should be prevented, or at least greatly reduced, owing to the novel coating. By means of the coating according to the invention, the probability of success of prostheses that are implanted without cement is therefore increased, because the risk of an infectious rejection reaction is decisively reduced.

The problem described is solved according to the invention, in that a defined metal content (in particular silver or metals with comparable action, for example zinc or copper, but also mixtures of these metals) is incorporated by various methods in the calcium phosphate layers produced by thermal spray technology. The term “calcium phosphate layers” means, in particular, hydroxyapatite (HA), α- and/or β-tricalcium phosphate (TCP), tetracalcium phosphate (TECP) or mixtures of these variants optionally with additions of calcium oxide. Without restricting the scope of the invention, other calcium phosphates can also be used, for example pyrophosphate or anhydrous oxyapatite, with and without addition of calcium oxide and/or fluoroapatite.

A first possibility for establishing the metal content employs the method of ion exchange in the starting powder for production of the implant coating using spray technology. In this, an established number of Ca ions in the crystal lattice is replaced e.g. with Ag ions according to U.S. Pat. No. 5,009,898 and/or incorporated at interstitial sites. Another possibility consists of carrying out the ion exchange only on the already prepared spray powder with the grain size distribution required for the thermal spraying technology. As a third possibility, pure, undoped calcium phosphates can also be used for the production of the metal-containing spray powder and these can for example be mixed with metallic silver powder. The sprayed layers produced according to the invention from the aforementioned spray powders, and containing Ag or other metals, are characterized in that for example the silver is in ionic form, and/or is distributed finely and uniformly in the layer in metallic form in concrete portions of material.

Antibacterial and osseointegrating calcium phosphate layers produced in this way by thermal spraying differ very characteristically from all other known antibacterial protective layers.

For their production according to the invention by spray technology, metal-containing calcium phosphate spray powders (for example with Ag) are to be prepared first, in each case with particle morphology, grain size and grain size distribution suitable for the various methods of thermal spraying. For example, densely sintered or agglomerated/sintered spray powders adjusted in grain size range to 10 to 200 μm, 30 to 150 μm or 45 to 125 μm, though preferably to 20 to 60 μm, or for especially fine layers and/or special spraying methods also to 5 to 25 μm, are recommended for the various spraying methods.

If calcium phosphates with a risk of phase transition (e.g. HA) are required completely or partially for production of the layer, it is recommended to use those thermal spraying methods that permit fast process speeds at lower particle temperatures and with very short dwell time of the powder particles above phase transition temperatures (e.g. VPS), in order to transfer as much as possible of the spray powder composition into the sprayed layer.

In the sense of the invention, powder or spray powder consists of a collection of individual grains with different dimensions and shapes, which are in their turn composed of finer particles, but can also be compact and homogeneous within themselves.

The term “carrier grains” means, in the sense of the invention, spray powder grains that contain or comprise at least one active particle. The term “filler grains” means, in the sense of the invention, powder grains or spray powder grains that do not comprise any active particle or particles.

### BRIEF DESCRIPTION OF THE DRAWINGS

The invention will now be explained on the basis of drawings and examples of application.

FIG. 1a shows a first variant of the metal/calcium phosphate spray powder particles required for production according to the invention, before passing through the thermal spray source, with Ag selected as the antibacterial metal.

FIG. 1b shows the Ag/calcium phosphate spray powder grain after passing through the thermal spray source and after impinging on the implant surface.

FIG. 2 shows a schematic sectional view of details of the layer structure for various examples of antibacterial, bioactive prosthesis coatings produced according to the invention.

FIG. 3 shows a schematic sectional view of a variant of the invention, preferably optimized for orthopedics.

FIG. 4 shows a schematic sectional view of a metal/calcium phosphate sprayed layer according to the invention, as preferably employed in traumatology.

FIG. 5 shows an implant according to the invention and details of the covering layer of this implant.

FIG. 6 shows a schematic representation of a thermal spraying system.

FIG. 7 shows a schematic representation of plasma spraying equipment, a component part of a thermal spraying system according to FIG. 6.

### DETAILED DESCRIPTION OF THE INVENTION

### Spray Powders

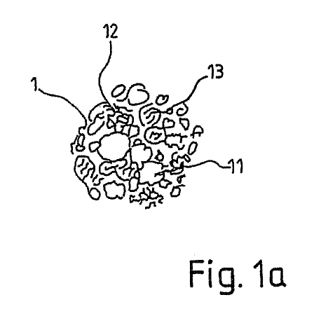

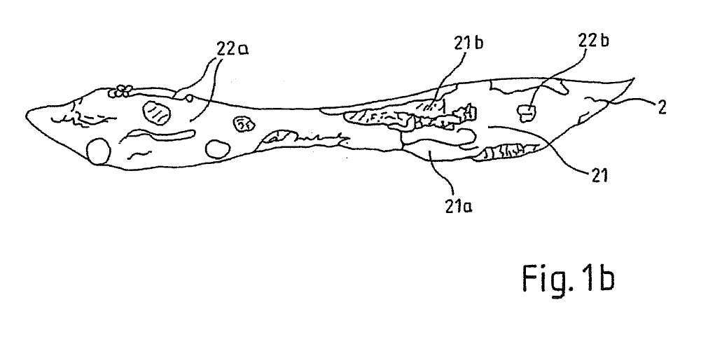

For the preparation of a first variant of spray powder for production of the thermally sprayed layer, according to the invention firstly calcium phosphate particles with size from about 0.1 to max. 5 μm, produced in a precipitation method, with or without antibacterial metal ions (e.g. Ag) in the molecule or at interstitial sites of the crystal lattice, are mixed homogeneously according to the prior art in the required ratio with antibacterial metal particles (for example Ag) of the same order of size, spray-dried, optionally sintered and fractionated in the desired grain size distribution. This results in metal/calcium phosphate spray powder grains according to FIG. 1a. Each powder grain 1 contains a number of metal particles (e.g. Ag) 12 determined by the mixture ratio, incorporated randomly and according to their size distribution in the composite of the baked calcium phosphate particle 11. The Ag particle fraction preferably has a lower limit, for clear demarcation from Ag incorporated atomically (as ion). In particular, Ag particles 12 with a diameter of less than 0.1 μm are excluded. It is preferable to use the fraction 0.5 to 5 μm, or for special applications also 1 to 10 μm. This means that there may even be Ag particles 12 in the spray powder grains that may be larger than the calcium phosphate particles 11.

An important feature of the metal-calcium phosphate spray powder required according to the invention (for example with Ag) is that two different calcium phosphate particles can be used for its production: on the one hand those that were doped with metal ions to replace calcium ions in defined numbers in the previous precipitation method 13, and on the other hand also with calcium phosphate particles without this doping with metal ions 11. It is thus possible to define the metal content of the spray powder in two size levels:
a) Of atomic size, incorporated as metal (Ag) ions in the calcium phosphate lattice and/or in the interstitial sites of the crystal lattice 13. In this case, for example the Ag content is established over a wide range on the basis of the concentration ratio of the Ag salt (for example Ag nitrate) in the aqueous solution of e.g. Na phosphate and Ca chloride in the respective mixture ratio for precipitation of the desired variant of calcium phosphate, observing prescribed reaction times and temperatures. Preferably the Ag content is set at between 0.05 and 2% relative to the calcium phosphate. The range from 800 ppm to 2000 ppm has proved particularly effective.
b) Of the size of actual portions of material as metal (Ag) particles 12, combined as a composite with the calcium phosphate particles 11 to form the spray powder grain. The metal content at this level can be varied over a wide range e.g. by means of the size and number of Ag particles (12) incorporated in the mixing process. The recommended range is set at 0.1 to 10%, preferably 0.5 to 2%.

On the one hand the total silver content of the spray powder must not be below the concentration at which the Ag content in the resultant sprayed layer no longer has antibacterial action. According to data in the literature this minimum concentration is about 30 mg/kg body weight for each patient. The task of the invention is of course to maintain the Ag concentration in the layer surface/bone bed active level after implantation without cement permanently above an effective minimum concentration for the required length of time. On the other hand, it is essential to ensure that the Ag content in the sprayed layer stays sufficiently far below the toxicity limit for human tissue. Again there is relevant information in the literature, recommending, depending on the test method and the patient's constitution, values from 0.5 to 1.2 g/kg body weight.

According to the invention, the total Ag content can additionally be finely adjusted by admixture of calcium phosphate grains without Ag content still in established proportions to the Ag-containing calcium phosphate spray powder. Their size is within the limits of the grain size distribution of the Ag-containing spray powder. Suitable mixture ratios of the spray powder fractions with and without Ag have been found to be 90 to 10%, preferably 70 to 30%. For long-term implants preferably 40 to 90% and in the case of spray powder for metal-containing sprayed layers in traumatology preferably 10 to 40%, with the percentages referring to the admixture of metal-free calcium phosphate. This admixture can take place either before spraying with an established mixture ratio in the spray powder itself or later during layer formation via a 2nd powder feed line to the thermal-energy free jet. There is then even the additional possibility of altering this mixture ratio during layer formation, e.g. to produce a layer with graduated metal content depending on layer thickness. In this way it is also possible to spray sandwich structures, in which each individual layer has a defined metal content. These variants will be described in more detail later, with reference to the use of the metal-containing calcium phosphate sprayed layers in traumatology and in orthopedics.

Another powder variant has also proved useful for producing the antibacterial calcium phosphate layer according to the invention by thermal spraying. Provision of the metal content at the atomic level by incorporating metal ions in the calcium phosphate crystal lattice and at interstitial sites does not take place until preparation of the calcium phosphate spray powder with the required grain size distribution for the thermal spraying process, namely by means of an absorption and immobilization process in a chemical solution. For example, 100 g of prepared calcium phosphate spray powder (with or without incorporated Ag particles (12)) in aqueous solution of 10 g of Ag nitrate in 1000 g of distilled H₂O is supplemented, for the specified time of action and bath temperature, with the required number of Ag ions. It has been found that the Ag concentration in the powder can be monitored by determining the content of Ca ions in the solution, as these were replaced by the Ag ions and consequently were released. An especially effective metal concentration with this powder variant has been found to be 500 to 2000 ppm Ag, preferably 1000 ppm.

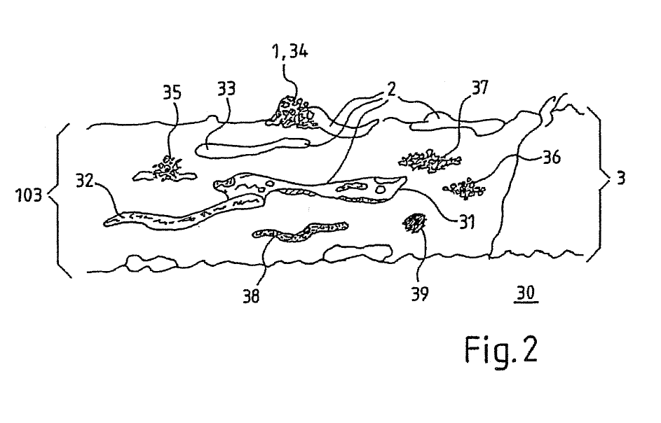

It is also conceivable, without limiting the invention with respect to the size of the spray powder grains, to mix metal particles into the calcium phosphate spray powder (FIG. 2), e.g. in order to incorporate it in the sprayed layer as a pure Ag-lamella 38. However, the size of the Ag particles preferably has an upper limit of 50 μm.

### Sprayed Layers

If the calcium phosphate powders produced according to the description are now transformed by thermal spraying into sprayed layers, with appropriate selection of the thermal energy each spray powder grain is converted according to FIG. 1a into a sprayed lamella (FIG. 1b) 2, flattened and spread out by the mechanical energy during impingement on the substrate surface. The individual calcium phosphate particles 11 form a homogeneous core 21, in which the metal particles 12 are incorporated depending on size either as melted fine lamellae 22a or unchanged in the original form 22b, but are also located on its free surface. The homogeneous core 21 itself is composed of metal-rich zones 21b and metal-free zones 21a, depending on whether there is a melted calcium phosphate particle with atomic metal content 13 or without atomic metal content 11 at the site in question.

A sprayed layer 3 (or covering layer 103) shown in FIG. 2, which is on an implant 30, is composed of a large number of individual sprayed lamellae 2, and the energy of the spraying process can optionally also be set so that a defined proportion of the metal-calcium phosphate powder is incorporated without conversion to a sprayed lamella, and thus in its original form as powder grain 1 in the sprayed layer 3. Preferably this relates to the larger spray powder particles in the selected powder fraction. Overall, the sprayed layers according to the invention can contain, depending on the choice of thermal spray energy and form of the starting powder, the following components at varying concentration and arranged in various levels:
a) Melted sprayed lamellae 31 (active lamella) with inclusions of melted-metal fine lamellae 22a and unmelted metal particles 22b, where the calcium phosphate fraction 21 on the one hand contains metal ions 21b, and on the other hand is metal-free 21a.
b) Melted sprayed lamellae 32 (active lamella) according to a) with metal ions in the calcium phosphate fraction 21b.
c) Melted sprayed lamellae 33, which contain neither metal ions nor actual metallic material fractions in the sprayed lamella. They are designated as filler lamellae, formed from filler grains.
d) Unmelted active grains 34, which correspond in their structure and composition to the starting powder 1, and thus contain metal particles 12, metal ions in the calcium phosphate fraction 13 and metal-free calcium phosphate 11.
e) Unmelted active grains 35 according to d) without metal particles 12 but instead with metal ions in the calcium phosphate fraction 21.
f) Unmelted active grains 36 according to d) with metal particles 12 but without metal ions in the calcium phosphate fraction 21.
g) Unmelted filler particles 37 without Ag, neither atomically nor as a concrete portion of material.

For particular applications of the metal-containing, thermally sprayed calcium phosphate layer, it may be advantageous to incorporate only the atomic form of the metallic inclusion in each individual grain of the spray powder by ion exchange. The sprayed lamellae then correspond to those of the pure calcium phosphate layer, the only difference being that, as in the spray powder, in each sprayed lamella individual Ca ions are replaced with metal ions and/or these are inserted at interstitial sites in the calcium phosphate lattice.

Addition of metal in the form of concrete portions of material takes place by means of metal-spray powder grains, corresponding in size to the lower range of the calcium phosphate spray powder grain distribution. If this is e.g. 20 to 50 μm, the metal grain fraction is preferably selected between 5 and 25 μm. The result is a metal-calcium phosphate layer 3 with large-area metal-sprayed lamellae (38), e.g. Ag-lamellae.

It should be noted, however, that large-area regions of pure metal are present in the sprayed layer, depending on grain size up to 100 μm in diameter and about 0.2 to 5 μm thick, preferably 20 μm in diameter and 1 μm thick. Some of the metal grains can also be incorporated as unmelted metal grains (39) in the calcium phosphate layer 3.

When metal is added during spray powder production it should be borne in mind that the thermal spraying process can lead to changes in the relative proportions between spray powder and sprayed layer, mainly when the spray powder is fused well for production of a dense layer structure. This effect can partly be compensated if the component that develops a higher vapor pressure in the molten state is incorporated during mixing in a grain size distribution for which the average particle size is displaced towards larger particles. Thus, comparing calcium phosphate (ceramic material) e.g. with silver (metal), based on the physical characteristics (melting and evaporation temperature, thermal conductivity and heat capacity) we should expect a decrease in atomic Ag content and an increase in Ag content in the form of concrete portions of material in the sprayed layer in comparison with the mixture ratio in the spray powder. The percentage concentration ratio Ag/calcium phosphate in the spray powder will therefore either increase in favor of Ag or decrease in the sprayed layer, depending on whether the Ag fraction is present in concrete portions of material in the spray powder, or as atomic (ionic) Ag fraction.

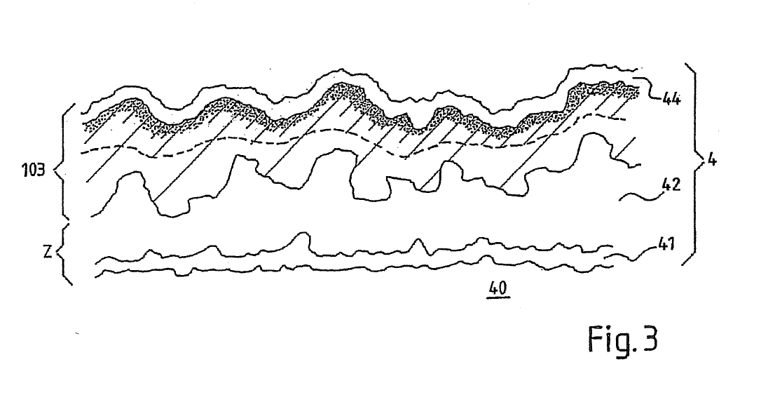

Further embodiments for production, according to the invention, of the calcium phosphate layer containing antiinflammatory, osseoinductive and antibacterial metal in defined form and concentration take into account important findings from the medical application of thermal spraying technology and are explained in more detail by means of FIG. 3, which shows a succession of layers 4 on an implant 40 intended for orthopedics.

On this implant 40, the adherence of the metal/calcium phosphate layer 103 sprayed on as a covering layer according to the invention e.g. in the VPS method is advantageously firstly ensured with a first sprayed layer of titanium 41, from about 20 to max. 50 μm thick, acting as an adhesive layer. Titanium is a known adhesion promoter and has very good anchorage with all surfaces of the materials used for the production of endoprostheses: metals (e.g. Ti), metal alloys e.g. CoCr, plastics e.g. PEEK with or without carbon fiber reinforcement and ceramics e.g. Al2O3, ZrO2 and mixed ceramics. All implant materials are preferably roughened by sandblasting before coating, to intensify the anchorage effect. This Ti adhesion layer 41 is essential for ceramic prostheses—merely roughening the surface by sandblasting does not produce the necessary adherence. A Ti adhesion layer should not be used for implants made of special steel. To make it possible to apply the metal/calcium phosphate layers 103 according to the invention on less biocompatible materials as well, e.g. on carbon fiber reinforced PEEK, the Ti layer 41, which was initially only sprayed on for the purpose of promoting adhesion, acts simultaneously as sealing of the substrate surface and thus brings about its conversion to a biocompatible implant.

Without interrupting the coating operation, the entire succession of layers of the metal/HA layer 103 is applied directly on the freshly sprayed Ti base layer (41). In this case the energy of the plasma free jet, into which the metal/HA active powder produced according to the invention is injected, is optionally set so that either the powder particles are melted completely and/or are only partially melted or fused. This specially controlled spraying process therefore makes it possible to produce the metal/HA layer optionally with high crystallinity or with a high proportion of amorphous structure. For particular applications it may also be advantageous to provide a graduated transition from crystalline to amorphous layer structure. This means that during the spraying process with continuous powder injection, the plasma-free jet energy is continually displaced for example from lower values to higher values, and consequently in the direction towards the layer surface, the structure of the layer becomes increasingly compact, but at the same time also more amorphous. In fact it is known from the literature that the solubility of HA, surrounded by human body tissue, increases with increasing proportion of amorphous layer. Therefore the metal/HA layer 103 produced according to the invention acquires a graduated solubility, which is higher in the initial phase immediately after implantation and decreases continuously with increasing dwell time in the body. On the one hand union of the prosthesis surface is promoted and accelerated by the dissolved calcium and phosphate ions. On the other hand the release of metal ions is also increased initially and therefore the antibacterial action is intensified in the critical phase immediately after implantation. With increasing coverage of the implant surface by newly formed bone tissue, less antibacterial metal is required for reliable prevention of infection. This effect of variable (increasing or decreasing) metal concentration in the bone/prosthesis composite depending on the dwell time can additionally also be ensured by varying, simultaneously with the increase in thermal energy in the plasma free jet, the admixture of metal-free calcium phosphate spray powder by means of additional powder injection either in stages (layer 103 as a sandwich structure as shown in FIG. 3) or continuously (layer 103 with graduated metal concentration, not shown schematically). It is expressly pointed out here that formation of the layer variants depicted is not limited to plasma spraying. It is also possible with the other methods of thermal spraying technology for the free jet energy and additional powder injection to be varied and included correspondingly.

For general support of the osseoconductive and osseoinductive action of the metal/calcium phosphate sprayed layer, a rough Ti layer structure 42 can additionally be sprayed on between the titanium adhesion layer 41 and the e.g. Ag/HA layer 103. Thus, in the example of application according to FIG. 3 there is an interlayer Z, consisting of 2 layers, the titanium adhesion layer 41 and the rough Ti layer structure 42. The grain fraction of the required Ti spray powder is selected so that the surface of the layer has a rough, open-pore configuration, which is especially favorable for the ingrowth of bone cells. The latter prefer an open surface porosity with pores in the range from 50 to 400 μm. The trick is that this surface structure of the Ti interlayer 42 spreads into the metal/calcium phosphate layer 103 that is deposited on it, and is thus leveled off just slightly, but increasingly with increasing thickness of the metal/calcium phosphate layer 103. Especially in this variant of the layer, therefore, the layer thickness of the metal/calcium phosphate layer 103 is limited to a maximum of 150 μm and is preferably in the range 30 to 100 μm. In the case of graduated crystallinity for example the first 20 to 60 μm of the antibacterial calcium phosphate layer should be highly crystalline. In the middle region of the layer from 50 to 120 μm the amorphous fraction should then increase continually and should then be highest in the final surface layer with a thickness from about 20 to max. 50 μm, e.g. should be at least 40 to 80%, preferably 55 to 70%.

Without limiting the invention it is also possible to spray an additional metal-free calcium phosphate layer 44 of high solubility onto the metal/calcium phosphate layer 103 produced according to the invention, e.g. a TCP layer, a highly amorphous HA layer or an HA/TCP mixed layer, limited in thickness to 10 to 60 μm, preferably about 20 to 40 μm thick, as this value leads to formation of a completely closed covering even with thermally sprayed layers. The range from 10/90 to 40/60% has proved suitable for the HA/TCP mixture ratio, the proportion of TCP preferably being higher, at 60 to 80%, when a thin covering layer (44), only about 10 to 20 μm thick, is used. Thus, in the example of application according to FIG. 3 there is a multilayered sprayed layer 4, which consists of the multilayered metal/calcium phosphate covering layer 103 optionally with graduated or sandwich structure, the covering layer 44 and the single-layer or two-layer interlayer Z (adhesion layer 41 and Ti structure 42).

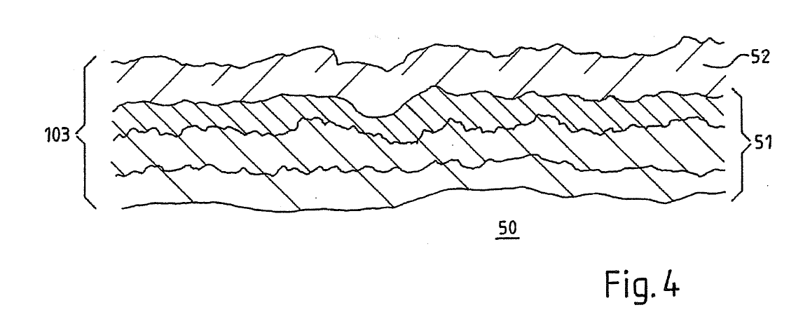

FIG. 4 shows another example of a covering layer (103) according to the invention, with the multilayered structure optimized for the traumatology application. It consists of a 1st layer of calcium phosphate 51 with variable proportion of amorphous structure and TCP content sprayed directly on the surface of an implant 50, constructed either as a sandwich structure or as a graduated succession of layers, with at least 10 to 40% TCP and 40 to 90% HA with a proportion of amorphous layer of at least 20 to 80%, preferably 50 to 60%. The thickness of this 1st layer 51 can optionally be 20 to 100 μm, preferably 40 to 60 μm. During spraying of this 1st layer 51 the thermal energy was selected so that about 70 to 100% of the spray powder grains were converted to melted sprayed lamellae, preferably about 80 to 90%. Once again the energy of the plasma free jet is varied, in contrast to the succession of layers 4 for orthopedics, but in the traumatology application so that the proportion of crystalline layer decreases in the direction towards the substrate surface. For this 1st layer 51, a metal-calcium phosphate powder was selected with a metal content that is only slightly above the limit of antibacterial action of 300 ppm, preferably 500 to 1150 ppm. On top of this 1st layer 51 there is a 2nd layer 52, only about 10 to 50 μm, preferably 20 to 30 μm thick. It differs from the 1st layer in having a higher metal content, at the same time with lower solubility. Ideally the new bone tissue that forms very quickly on the coating owing to the osseoconductive/osseoinductive action takes about ¾ of the envisaged implantation time to dissolve this 2nd layer 52 of the complete coating and/or to transform it to further bone tissue. After that, the new bone tissue is in direct contact with the still-present 1st layer 51 of the metal/calcium phosphate succession of sprayed layers. This is now also dissolved very quickly and/or converted to bone tissue in the remaining ¼ of the implantation time. The increasing content of TCP towards the surface of the prosthesis and/or amorphous HA in the 1st layer 51, which leads to increasing solubility, is responsible for this. As a result, the newly formed bone tissue comes directly into contact with the prosthesis surface, which is smooth and has an average roughness of well below 2 μm, preferably 0.1 to 1 μm. With this surface configuration, there is formation of a zone of connective tissue between the bone tissue and the prosthesis surface, which facilitates the planned explantation of this trauma prosthesis after the planned dwell time and completion of healing of the bone fracture. According to the example of application shown in FIG. 4, a covering layer 103 interlayer (adhesion layer and/or Ti structure) is thus sprayed directly on the implant 50.

### EXAMPLES

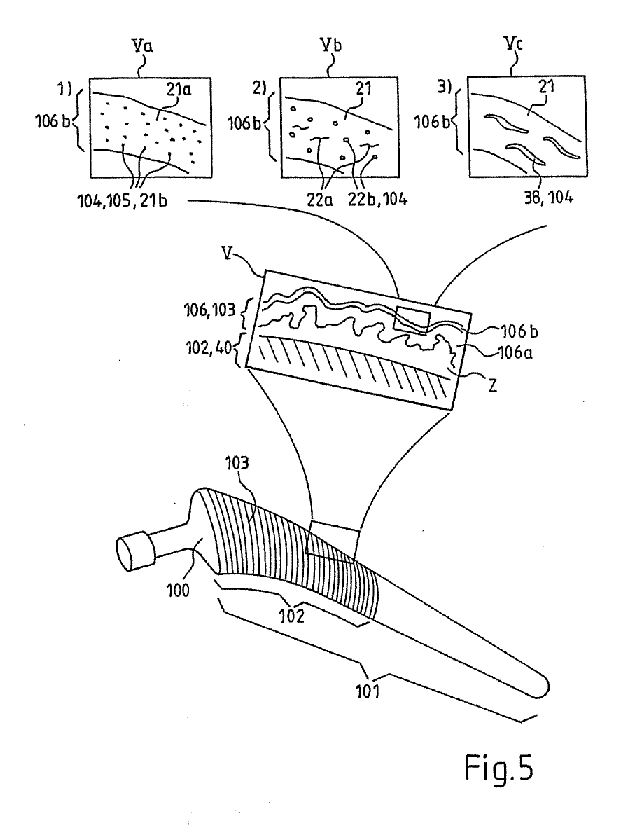

FIG. 5 shows, as a practical example of application for a coated implant in orthopedics, a femoral shaft 100, usually made of titanium alloy. Part 101 serves as the anchoring region in a thigh bone (not shown). This anchoring region of the femoral shaft is either complete, or as shown in FIG. 5, coated in a partial region 102 with the covering layer 103 produced by thermal spraying technology. In the partial region 102, therefore, the femoral shaft 100 forms the substrate surface 40 for the covering layer 103, in the example shown with interlayer Z (adhesion layer 41 and additional Ti layer structure 42) sprayed on. The adhesion layer 41 forms, together with the Ti layer structure 42, a two-layer interlayer Z. For clarity it should be mentioned that the interlayer Z is underneath the covering layer 103 and on the substrate material 40. In FIG. 5, a window V shows an enlarged view of a basic structure of the covering layer 103, which is on the interlayer Z. According to the representation in the window V, the substrate material 40 is followed by the two-layer interlayer Z made of titanium. This titanium interlayer Z is, as already mentioned, not necessarily provided, but is used whenever optimized adhesion of the subsequently applied calcium phosphate layer 103 is desired or whenever increased roughness is desired relative to the subsequently applied calcium phosphate layer 103. The subsequently applied calcium phosphate layer 106 (corresponding to 103) is formed, according to a first embodiment, by a calcium phosphate layer 106a without antibacterial action and next a calcium phosphate layer 106b with active constituents 104 against bacteria. According to a second embodiment (not shown), the calcium phosphate layer 103 consists exclusively of a calcium phosphate layer 106b with active constituents 104 against bacteria, and accordingly with internal structure corresponding to FIG. 3. In general, for example the following variants are envisaged for the structure of the covering layer 103:

Variant 1: The covering layer 103 consists of a calcium phosphate layer 106a without antibacterial action and a calcium phosphate layer 106b with active constituents 104 against bacteria, with the calcium phosphate layer 106b representing the outermost layer that comes in contact with the bone or tissue.

Variant 2: The covering layer 103 consists only of a calcium phosphate layer 106b with active constituents 104 against bacteria, with the calcium phosphate layer 106b also representing the outermost layer that comes in contact with the bone or tissue.

Variant 3: The covering layer 103 consists of a first layer 106a without antibacterial action, a 2nd layer 106b with antibacterial action, followed by another calcium phosphate layer 44 without antibacterial action, formed to have high solubility.

Three examples of the structure of the calcium phosphate layer 106b with active constituents 104 against bacteria are shown schematically in windows Va to Vc.

According to a first embodiment Va, the calcium phosphate layer 106b is constructed so that the active constituents 104 are incorporated as metal ions 105 (shown magnified) in calcium phosphate 21b, which together with the metal-free calcium phosphate 21a form the layer. In this case the metal ions 105 are present for example as Ag⁺ ions and/or as Cu²⁺ ions, or as mixtures of both.

According to a second embodiment Vb, the calcium phosphate layer 106b is constructed so that metal particles 22a and/or 22b, which form the active constituents 104, are incorporated, homogeneously distributed, in calcium phosphate 21. In this case the melted and/or unmelted active constituents 104 are present for example as Ag particles and/or as Cu particles.

According to a third embodiment Vc, the calcium phosphate layer 106b is constructed so that lamellae 38 of metal form the active constituents 104, which are incorporated in calcium phosphate 21. In this case these antibacterial metal lamellae 38 consist for example of Ag and/or Cu or of Zn or of mixtures of these antibacterial metals.

According to the foregoing and the detailed information on the production of the spray powder and sprayed layers according to the invention, mixed forms of embodiments Va to Vc are also envisaged as further embodiments, e.g. in which the structure of the calcium phosphate layer 106b varies in the direction towards the substrate material 40, for example wherein the form of the active constituents 104 is varied, and/or wherein the concentration of the active constituents 104 increases or decreases.

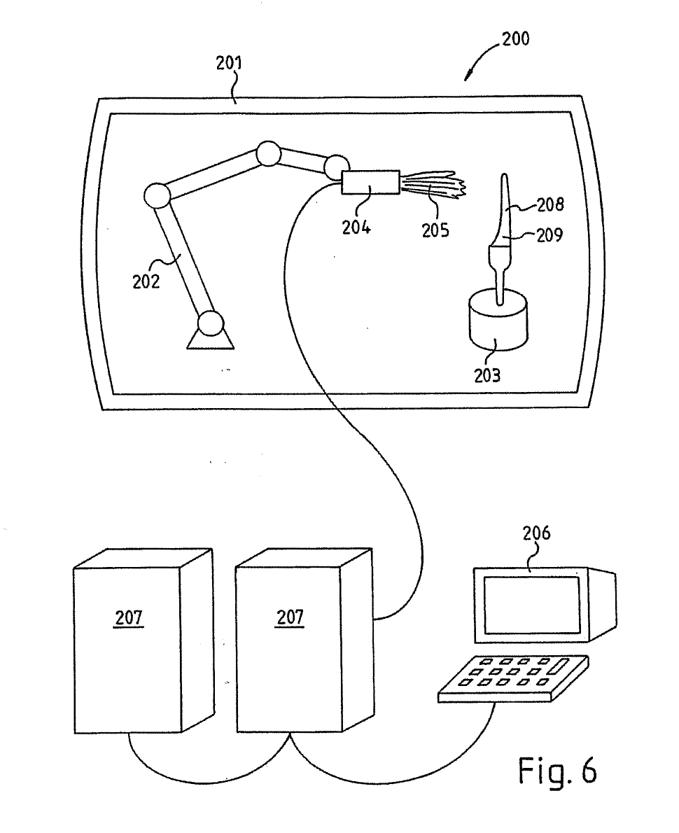

FIG. 6 is a schematic representation of a thermal spraying system 200, which is employed for producing an implant according to the invention or with which the method according to the invention can be carried out. The spraying system 200 comprises a vacuum chamber or a soundproof booth 201, in which a robot 202 and a holding device 203 are arranged. The robot 202 guides a torch 204, in which a plasma jet 205 can be produced. The spraying system 200 further comprises an operating unit 206, with which in particular the robot 202 and the torch 204 can be controlled and adjusted. Furthermore, the spraying system 200 also comprises the usual peripherals 207, for example cooling system, switch cabinet, container for spray material and the like. An uncoated implant 208 is held in the holding device 203, which after spraying on a special covering layer can be removed from the spraying system 200 as a coated implant 209 according to the invention.

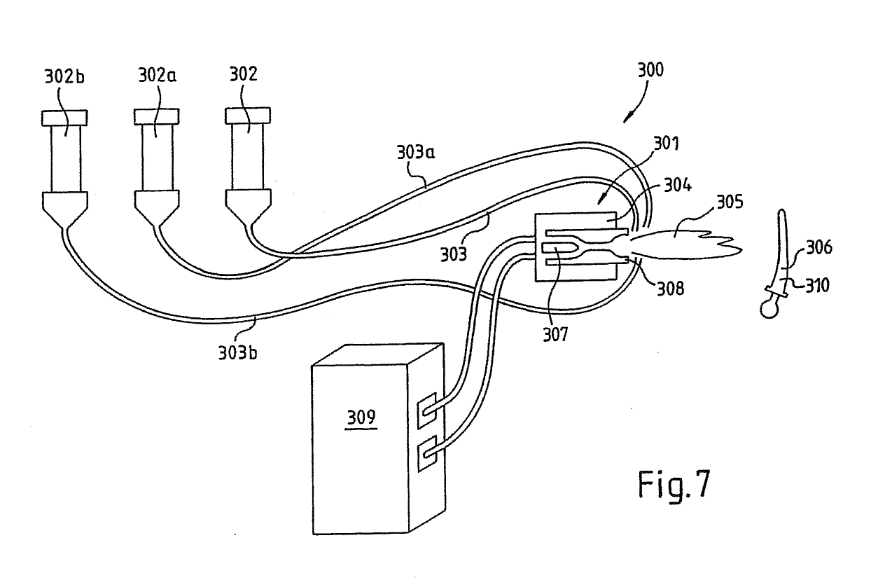

Plasma spraying equipment 300, which can be used for example in the thermal spraying system shown in FIG. 6, is shown schematically in FIG. 7. The plasma spraying equipment 300 comprises a torch 301, which is also called a plasmatron. The latter is supplied by one or more powder feeders 302, 302a and 302b with powder 303, 303a and 303b with composition according to the invention, and with the plasma jet 305 discharged from a nozzle 304, is deposited on the implant 306 that is to be coated. Nozzle 304 essentially comprises a cathode 307 and an anode 308. Torch 301 is connected, for power supply and cooling, to a supply module 309. After plasma coating with the corresponding powders 303, 303a and 303b, the implants 310 produced according to the invention then have antibacterial properties, the spray powders being injected individually in succession or simultaneously with variable proportions in the plasma free jet.

### LIST OF REFERENCE SYMBOLS

1 powder grain
2 sprayed lamella
3 sprayed layer
4 succession of layers
11 calcium phosphate particle
12 Ag particle
13 calcium phosphate particle with Ag ions
21 homogeneous core, calcium phosphate fraction
21a Ag-free calcium phosphate zone
21b Ag-rich calcium phosphate zone
22a melted fine lamella of Ag
22b unmelted Ag particle in original form
30 implant
31 melted sprayed lamella (1st variant)
32 melted sprayed lamella (2nd variant)
33 melted sprayed lamella (3rd variant)
34 unmelted spray powder grain (active grain) (1st variant)
35 unmelted spray powder grain (active grain) (2nd variant)
36 unmelted spray powder grain (active grain) (3rd variant)
37 unmelted spray powder grain (filler grain)
38 melted Ag sprayed lamella
39 unmelted Ag spray powder grain
40 implant
41 Ti adhesion layer
42 rough Ti layer structure
44 Ag-free calcium phosphate layer
Z interlayer, consisting only of 41, or of 41 and 42
50 implant
51 1st layer
52 2nd layer
100 implant
101 anchoring region
102 coated anchoring region
103 covering layer
104 active constituent
105 metal ions, e.g. Ag⁺, Cu²⁺ or Zn²⁺
106 calcium phosphate layer
106a calcium phosphate layer without antibacterial action
106b calcium phosphate layer with active constituents
200 thermal spraying system
201 vacuum chamber or soundproof booth
202 robot
203 holding device
204 torch
205 plasma jet
206 operating unit
207 peripherals
208 uncoated implant
209 coated implant
300 plasma spraying equipment
301 torch, plasmatron
302,a,b spray powder in the powder feeders
303,a,b powder lines
304 nozzle
305 plasma jet
306 implant yet to be coated
307 cathode
308 anode
309 supply module
310 implant with antibacterial properties
V window
Va-Vc windows

We claim:

1. An implant of biocompatible materials, in particular prosthesis implanted without cement for traumatology and/or orthopedics, comprising a main body with an anchoring region which anchors in bone or tissue, the anchoring region being provided at least partially with a covering layer, the covering layer being formed from a powder using a thermal spraying method in particular a plasma spraying method, wherein the powder consists essentially of calcium phosphate and comprises antibacterially effective active constituents.

2. The implant as claimed in claim 1, wherein the covering layer of the anchoring region that contacts with bone or tissue is applied directly on the main body or with inclusion of at least one laminated interlayer on the main body, in particular in a region.

3. The implant as claimed in claim 1, wherein the antibacterially effective active constituents of the covering layer consist of metal and/or precious metal, in particular comprise silver and/or copper and/or zinc and mixtures thereof and in that the active constituents can in particular be in atomic and/or ionic form and/or integrated in the crystal lattice and/or as concrete portions of material.

4. The implant as claimed in claim 1, wherein the active constituents in concrete portions of material are incorporated in the covering layer preferably either unmelted as particles or spray powder grains with a diameter from 0.5 to preferably 25 micrometers and/or as melted sprayed lamellae.

5. The implant as claimed in claim 1, wherein the powder for the production of the covering layer consists essentially of calcium phosphate, in particular of hydroxyapatite and contains a defined proportion of active constituent of metal and/or precious metal, preferably silver and/or copper and/or zinc and/or mixtures thereof, the metal content being in atomic and/or ionic form and/or mixed in as a concrete portion of material.

6. The implant as claimed in claim 1, wherein the powder, a defined number of Ca ions in the crystal lattice of the calcium phosphate are replaced by ion exchange with metal ions, in particular Ag ions and/or metal atoms are incorporated at interstitial sites.

7. The implant as claimed in claim 1, wherein the ion exchange and/or the incorporation of metals take place during precipitation of the desired calcium phosphate from aqueous solution by adding metal salts in an established ratio to the components required for the precipitation of calcium phosphate, with the number of metal ions incorporated and/or metal atoms intercalated being determined taking into account specified reaction times and temperatures.

8. The implant as claimed in claim 1, wherein ion exchange and/or the intercalation of metal atoms is carried out on the prepared calcium phosphate spray powder grain, which is in the optimized distribution for the selected thermal spraying method for production of the covering layer.

9. The implant as claimed in claim 1, wherein the calcium phosphate spray powder consisting of filler grains and/or active grains is mixed with metal grains, which are adjusted to the grain size distribution of the calcium phosphate powder and preferably are formed not smaller than 5 μm and not larger than 25 μm.

10. The implant as claimed in claim 1, wherein the release of the antibacterially effective metal ions can be controlled via the solubility of the calcium phosphate.

11. A method for coating an implant or a part of an implant, in particular a prosthesis that is implanted without cement for traumatology and/or orthopedics, with an antibacterially effective covering layer, formed from a powder using a thermal spraying method, wherein the powder comprises antibacterially effective active constituents and the thermal energy of a free jet produced by thermal spraying methods is adjusted during production of the covering layer to the melting point of the particles of the powder in such a way that the covering layer is formed from completely melted and/or only partially melted and/or still unmelted active constituents in particular with or without melted and/or partially melted and/or unmelted filler grains.

12. The method as claimed in claim 11, wherein the concrete portions of metal matching the calcium phosphate spray powder granulometry are only incorporated in the covering layer during layer production via a second, separate powder line, partially in the form of melted sprayed lamellae and/or unmelted in the original form.

13. The method as claimed in claim 11, wherein the thermal energy for production of the free jet controls the proportion of crystalline and/or amorphous constituents of the layer and hence the release of the active constituents.

14. The method as claimed in claim 11, wherein the covering layer is formed, depending on its layer thickness, with varying composition of active constituents and filler constituents.

15. The method as claimed in claim 11, wherein the thermal spraying method is carried out as a plasma spraying method.

16. A covering layer, in particular for an implant, wherein the calcium phosphate fraction of the layer has a set ratio of crystalline to amorphous layer structure, with the ratio varying depending on the thickness of the covering layer and thus having graduated solubility.

17. The covering layer as claimed in claim 16, wherein its graduated solubility is higher in the direction towards the layer surface and is lower in the bottom layers.

18. The covering layer as claimed in claim 17, wherein its graduated solubility is lower in the direction towards the layer surface and is higher in the bottom layers.

19. The covering layer as claimed in claim 16, wherein an additional calcium phosphate sprayed layer without active constituents and with high solubility is formed as a TCP layer or highly amorphous HA layer or as TCP/HA mixed layer.

20. The covering layer as claimed in claim 16, wherein active constituents in the spray powder are established so that the covering layer produced therefrom by thermal spraying technology on the one hand has sufficiently good antibacterial action, and on the other hand only releases an amount of metal ions and/or metal atoms that definitely do not have a toxic action, during the envisaged dwell time in the bone tissue.
